(19) United States
(12) Patent Application Publication
(10) Pub. No.: US 2010/0262018 A1
(43) Pub. Date: Oct. 14, 2010

(54) METHOD OF CONTROLLING A DEVICE FOR IMAGING THE INTERIOR OF TURBID MEDIA, DEVICE FOR IMAGING THE INTERIOR OF TURBID MEDIA AND COMPUTER PROGRAM PRODUCT

(75) Inventors: Levinus Pieter Bakker, Shanghai (CN); Bernhard J. Brendel, Hamburg (DE); Rik Harbers, Zurich (CH); Martinus Bernardus Van Der Mark, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/740,145

(22) PCT Filed: Oct. 28, 2008
(86) PCT No.: PCT/IB2008/054439

§ 371 Date: Apr. 28, 2010

(30) Foreign Application Priority Data
Nov. 5, 2007 (EP) 07119956.6
Jun. 4, 2008 (EP) 08157553.2

(51) Int. Cl. A61B 6/00

(52) U.S. Cl. 600/476

(57) ABSTRACT

A method of controlling a device for imaging the interior of turbid media is provided. The device comprises: a receiving portion (2) for receiving a turbid medium (1) to be examined; at least one light source (6) optically connected to the receiving portion (2) for irradiating the interior of the receiving portion (2); and at least one detector (7) optically connected to the receiving portion (2) for detecting light emanating from the interior of the receiving portion (2). The at least one light source (6) and the at least one detector (7) areoptically connected to the receiving portion (2) such that a plurality of different source-detector position combinations are formed over a complete measurement. The different source-detector position combinations define different light paths through the receiving portion (2). The method comprises a fast-measurement step in which a reduced set of data corresponding to only a part of the plurality of source-detector position combinations is generated for providing fast-information about the interior of the receiving portion (2).

### FIELD OF INVENTION

The present invention relates to a method of controlling a device for imaging the interior of turbid media, to a device for imaging the interior of turbid media, and to a computer program product.

### BACKGROUND OF THE INVENTION

In the context of the present application, the term turbid medium is to be understood to mean a substance consisting of a material having a high light scattering coefficient, such as for example an intralipid solution or biological tissue. Further, light is to be understood to mean electromagnetic radiation of a wavelength in the range from 400 nm to 1400 nm. The term “optical properties” covers the reduced scattering coefficient μ'ₛ and the absorption coefficient μₐ. Furthermore, “matching optical properties” is to be understood as having a similar reduced scattering coefficient μ'ₛ and a similar absorption coefficient μₐ.

In recent years, several methods and devices for examining turbid media, e.g. female breast tissue, have been developed. In particular, new devices for detection and analysis of breast cancer have been developed and existing technologies have been improved. Breast cancer is one of the most occurring types of cancer: in 2002, for example, more that 1.1 million women were diagnosed and over 410.000 women died of breast cancer world-wide. Several types of devices for imaging the interior of a turbid medium by use of light have been developed. Examples for such devices are mammography devices and devices for examining other parts of human or animal bodies. A prominent example for a method for imaging the interior of a turbid medium is Diffuse Optical Tomography (DOT). In particular, such devices are intended for the localization of inhomogeneities in in vivo breast tissue of a part of a breast of a female human body. A malignant tumor is an example for such an inhomogeneity. The devices are intended to detect such inhomogeneities when they are still small, so that for example carcinoma can be detected at an early stage. A particular advantage of such devices is that the patient does not have to be exposed to the risks of examination by means of ionizing radiation, as e.g. X-rays.

WO 00/56206 A1 discloses a device for imaging the interior of a turbid medium by using a light source to irradiate the turbid medium and photodetectors for measuring a part of the light transported through the turbid medium. A control unit is provided for reconstructing an image of the interior of the turbid medium on the basis of the measured intensities. The disclosed device is particularly adapted for examining female breasts. In order to allow the examination of the turbid medium, the device is provided with a receptacle as a receiving portion enclosing a measuring volume and arranged to receive the turbid medium. The light used for examining the turbid medium has to be transmitted from the light source to the turbid medium and from the turbid medium to the photodetectors. Due to different sizes of the turbid media to be examined, the size of the receptacle for receiving the turbid medium does not perfectly match the size of the turbid medium, i.e. a space remains between the receptacle and the turbid medium. A number of light paths coupling to the light source and a number of light paths coupling to photodetectors are distributed across the wall of the receptacle, i.e. ends of optical fibers acting as light guides are connected to the wall of the receptacle. The light source subsequently irradiates the turbid medium from different directions and the photodetectors measure a part of the light transmitted through the turbid medium. A plurality of such measurements are performed with the light directed to the turbid medium from different directions and, based on the results of the measurements, the control unit reconstructs the image of the examined turbid medium.

An optical matching medium for conducting optical energy generated by a light source at least from the light source to a turbid medium to be irradiated with at least a part of the optical energy generated by the light source is known from U.S. Pat. No. 5,907,406. The known optical matching medium can be used for imaging an interior of a turbid medium, such as biological tissue, using diffuse optical tomography. In medical diagnostics the matching medium may be used, for instance, for imaging an interior of a female breast. In that case, at least a part of the turbid medium, in this case a female breast, may be accommodated in a receiving volume.

In U.S. Pat. No. 5,907,406 the receiving volume is bounded by a cuplike wall portion. However, this is not always necessary. Inside the receiving volume, the part of the turbid medium under investigation is surrounded by the matching medium. Light from a light source is coupled into the receiving volume and into the turbid medium. The light is chosen such that it is capable of propagating through the turbid medium. For imaging an interior of a female breast, light having a wavelength within a range of 400 nm to 1400 nm is typically used. Scattered light emanating from the turbid medium as a result of coupling light into the receiving volume is coupled out of the receiving volume. Light coupled out of the receiving volume is used to reconstruct an image of an interior of the turbid medium. The matching medium is chosen such that the optical parameters of the matching medium, such as the absorption and scattering coefficients, are substantially identical to the corresponding optical parameters of the turbid medium. In this way, image artifacts resulting from optical boundary effects that occur when light is coupled into and out of the turbid medium can be reduced. Furthermore, use of the matching medium prevents the occurrence of an optical short-circuit in the receiving volume around the turbid medium. An optical short-circuit occurs when light is detected that has propagated along a path inside the receiving volume but outside the turbid medium and, as a consequence, has not been sufficiently scattered and attenuated. In that case the intensity of the insufficiently scattered and attenuated detected light may dwarf the intensity of detected light that has been scattered and attenuated through passage through the turbid medium.

In the known devices for imaging the interior of turbid media comprising a receiving portion receiving the turbid medium and an optically matching fluid for filling a space between the turbid medium and the receiving portion, sometimes the problem occurs that the space between the turbid medium and the receiving portion, e.g. a cup-like receptacle, is not completely filled by the optically matching medium. For example, if not enough optically matching medium is present, a gas pocket, which in most cases will be an air pocket, is present in the upper part of the space between the receiving portion and the turbid medium. Further, in particular if the optically matching medium is an optically matching fluid, gas bubbles may be present in the upper part of the space. If such undesired inhomogeneities are present in the space between the turbid medium and the receiving portion during a measurement for imaging the interior of a turbid medium, artifacts in the reconstructed image and serious measurement errors follow. The presence of the undesired inhomogeneities only becomes clear after the measurement and evaluation of the results. As a consequence, the time-consuming measurement has to be performed again to achieve images with high accuracy. As is evident, this forms an inconvenience and makes the imaging of the interior of the turbid medium expensive.

New approaches for further enhancing the accuracy of methods for detecting breast cancer by use of light have been made. For example, a fluorescent dye has been developed which can be injected into the body and will accumulate in cancer cells. The fluorescent dye acts as a contrast agent. If this fluorescent dye then becomes excited with light of a suitable wavelength, the locally emitted light can be detected. Based on the emitted light, size and localization of carcinoma can be determined. Thus a powerful method for detection and localization of breast cancer is provided. The dynamic behavior of the fluorescent contrast agent in the examined turbid medium, i.e. wash-in and wash-out of the contrast agent, provides additional information on top of the static information provided by a fluorescence image at a certain time-point. However, the relevant time constants of this dynamic behavior can be in the order of minutes necessitating fast measurements. Measurements for imaging the interior of turbid media in conventional devices require a large time interval and, thus, are not suited for determining the additional information which can be gathered from the dynamical behavior of the contrast agent.

### SUMMARY OF THE INVENTION

It is an object of the present invention to provide a method, a device and a computer program product enabling detection of undesired inhomogeneities in the receiving portion in a fast and efficient way and enabling determination of the dynamic behavior of a contrast agent in a turbid medium to be examined. Measurements for imaging the interior of turbid media with undesired inhomogeneities present in the space between the turbid medium to be examined and the receiving portion shall be prevented. It shall be ensured that actual measurements for imaging the interior of turbid media do not contain artifacts and measurement errors resulting from undesired inhomogeneities.

This object is solved by a method according to claim 1. A method of controlling a device for imaging the interior of turbid media is provided. The method is for such a device which comprises: a receiving portion for receiving a turbid medium to be examined; at least one light source optically connected to the receiving portion for irradiating the interior of the receiving portion; and at least one detector optically connected to the receiving portion for detecting light emanating from the interior of the receiving portion; the at least one light source and the at least one detector being optically connected to the receiving portion such that a plurality of different source-detector position combinations defining different light paths through the receiving portion are formed over a complete measurement. The method comprises a fast-measurement step in which a reduced set of data corresponding to only a part of the plurality of source-detector position combinations is generated for providing fast-information about the interior of the receiving portion.

Since the fast-measurement is performed in which only a reduced set of data is generated, inhomogeneities can be detected fast and efficiently before an actual measurement is performed. As a consequence, if inhomogeneities are present, they can be removed before the actual measurement is started and the time-consuming actual measurement is not performed with undesired inhomogeneities present. Thus, actual measurements containing artifacts and measurement errors caused by such inhomogeneities can be prevented. Further, since the fast-measurement uses only a part of the plurality of source-detector position combinations, the fast-measurement step can be performed fast which enables determination of the dynamic behavior of a fluorescent contrast agent present in the turbid medium.

Preferably, in the fast-measurement step, the intensity of light detected by the at least one detector is compared to an expected intensity and, based on this comparison, it is determined whether undesired inhomogeneities are present in the receiving portion or not. In this case, an easy and quick method of determining whether undesired inhomogeneities are present or not is provided. For this method, the fact is used that the relevant occurring inhomogeneities lead to large differences between the measured intensity and the expected intensity.

According to an aspect, a graphical representation of the comparison between the detected intensity and the expected intensity is provided to an operator of the device for imaging the interior of turbid media. As a consequence, the operator can easily determine whether undesired inhomogeneities have occurred or not and, in case such inhomogeneities have occurred, can perform appropriate actions to remove the inhomogeneities before the actual measurement is started. If no such inhomogeneities are identified, an actual measurement can be started.

According to another aspect, the graphical representation is provided such that it indicates at which position in the receiving portion an undesired inhomogeneity is present. In this case, the operator can remove inhomogeneities even faster, since information about the position of the inhomogeneity is provided.

If the fast-measurement step is performed to determine the dynamic behavior of a contrast agent located in the turbid medium, additional information about the interior of the examined turbid medium is provided.

Preferably, a plurality of fast-measurement steps generating reduced sets of data is performed at different times. In this case, the dynamic behavior of a contrast agent can be determined accurately.

The object is further solved by a device for imaging the interior of turbid media according to claim 7. The device comprises: a receiving portion for receiving a turbid medium to be examined; at least one light source optically connected to the receiving portion for irradiating the interior of the receiving portion; and at least one detector optically connected to the receiving portion for detecting light emanating from the interior of the receiving portion. The at least one light source and the at least one detector are optically connected to the receiving portion such that a plurality of different source-detector position combinations which define different light paths through the receiving portion are formed over a complete measurement. The device further comprises a control unit which is adapted to control the device such that: a reduced set of data corresponding to only a part of the plurality of source-detector position combinations is generated in a fast-measurement step for providing fast-information about the interior of the receiving portion. Thus, inhomogeneities can be detected fast and efficiently. Further, they can be reliably removed before an actual measurement is started. The dynamic behavior of a contrast agent present in the turbid medium can be determined.

According to an aspect, the receiving portion comprises a plurality of light guides for optically connecting to the at least one detector and to the at least one light source. The control unit is adapted such that, during the fast-measurement step, a reduced number of these light guides is used for subsequently directing light into the interior of the receiving portion. As a result, the light has to be switched for a reduced number of times during the fast-measurement as compared to an actual measurement, since less light guides are used during this step. Further, a reduced amount of data is generated. Thus, the fast-measurement can be performed time-efficient.

Preferably, the control unit is adapted such that, in the fast-measurement step, only light guides located in an upper part of the receiving portion are used for directing light into the receiving portion. According to a further aspect, the plurality of light guides are arranged on the receiving portion in a ring-like structure comprising a plurality of rings or sections thereof located in planes perpendicular to a vertical axis, and the control unit is adapted such that, in the fast-measurement step, only upper ones of the rings of light guides are used for directing light into the receiving portion. In this case, the fact is used that the relevant undesired inhomogeneities will accumulate in the upper region of the receiving portion. The presence of such inhomogeneities can be reliably determined even with a reduced data set. Further, the ring-like structure comprises a high symmetry which simplifies analyzing the sampled data.

If the control unit is adapted such that, during the fast-measurement, only the topmost ring of light guides is used for directing light into the receiving portion, the presence of inhomogeneities can be reliably determined with an extremely small amount of data generated during the fast-measurement. Thus, inhomogeneities can be identified very efficiently.

According to an aspect, a switch element is provided for subsequently directing light from the at least one light source into the light guides. Ends of the light guides are located in the switch element. Ends of said light guides which are used during the fast-measurement step are arranged adjacent to each other without ends of light guides which are not used in the fast-measurement step interposed therebetween. Thus, switching and redirecting of light in the fast-measurement step can be performed very fast. The time required for the fast-measurement step is further reduced.

Preferably, the device is a medical image acquisition device.

The object is further solved by a computer program product for a device for imaging the interior of turbid media according to claim 15. The computer program product is for a device which comprises: a receiving portion for receiving a turbid medium to be examined; at least one light source optically connected to the receiving portion for irradiating the interior of the receiving portion; at least one detector optically connected to the receiving portion for detecting light emanating from the interior of the receiving portion; the at least one light source and the at least one detector being optically connected to the receiving portion such that a plurality of different source-detector position combinations defining different light paths through the receiving portion (2) are formed over a complete measurement; and a control unit controlling the operation of the device. The computer program product is adapted such that, when it is loaded in the control unit of the device for imaging the interior of turbid media, the device is controlled such that: a fast-measurement step is performed in which a reduced set of data corresponding to only a part of the plurality of source-detector position combinations is generated for providing fast-information about the interior of the receiving portion. The computer program product realizes the same advantages as described with respect to the method. Further, the computer program product enables implementing the fast-measurement in known devices for imaging the interior of turbid media which comprise an appropriate control unit. Thus, the steps can be realized in known devices without requiring additional technical measures.

### BRIEF DESCRIPTION OF THE DRAWINGS

Further features and advantages of the present invention will arise from the detailed description of embodiments with reference to the enclosed drawings.

FIG. 1 schematically shows a receiving portion of a device for imaging the interior of turbid media with a turbid medium and an optically matching medium located therein;

FIG. 2 schematically shows the optical connection of the receiving portion and the light source and detector, respectively.

FIG. 3 schematically shows the receiving portion of FIG. 1 for the case that undesired inhomogeneities are present between the turbid medium and the receiving portion.

### DETAILED DESCRIPTION OF EMBODIMENTS

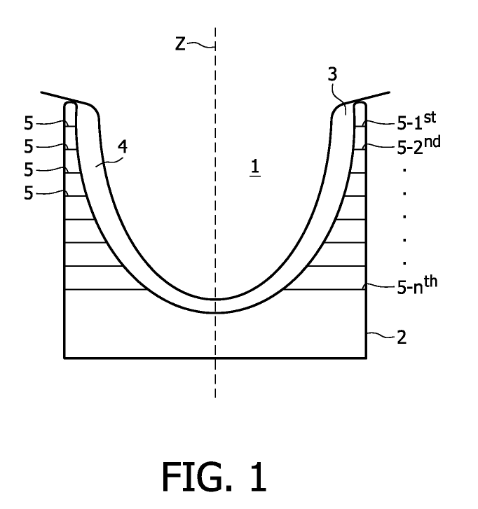

An embodiment of the present invention will now be described with reference to FIGS. 1 to 3. In the embodiment, the device for imaging the interior of a turbid medium is formed by a device for diffuse optical tomography (DOT), in particular by a mammography device. Since the overall construction of such a device is known to a skilled person, no detailed description of the device will be given.

In the device of the embodiment, the turbid medium 1 to be examined is a female human breast. The device is provided with a cradle on which a patient is placed. In the cradle, a receiving portion 2 enclosing a measuring volume and arranged to receive the turbid medium 1 is provided, as schematically indicated in FIG. 1. The receiving portion 2 has a cup-like shape with rotational symmetry with respect to a vertical axis Z and is provided with an opening 3. As can be seen in FIG. 1, the turbid medium 1 to be examined, i.e. the breast, is placed in the receiving portion 2 such that it freely hangs in the receiving portion 2 from the side of the opening 3. The inner surface of the receiving portion 2 facing the turbid medium 3 is provided with a plurality of ends of light guides 5 formed by optically guiding fibers connecting to a light source 6 and to a plurality of detectors 7. These ends of the light guides 5 are distributed on the inner surface of the receptacle 2 such that the receptacle 2 provided with the light guides 5 still comprises substantially rotational symmetry. The ends of the light guides 5 on the side of the receiving portion 2 are arranged on a plurality of rings 5-1^{st}, 5-2^{nd}, . . . , 5-n^{th} which are positioned in planes perpendicular to the axis Z, as schematically indicated in FIG. 1. On each ring, ends of a plurality of light guides 5 are distributed about the circumference of the receiving portion 2.

For example, in the device according to the embodiment 512 light guides 5 are provided the ends of which are distributed on the receiving portion 2. The light guides 5 may be formed by optically guiding fibers. In the embodiment, half of these light guides 5 are connected to an array of detectors 7. The other half of the light guides 5 is connected to a switch 9 capable to direct light from the light source 6 in either one of the 256 light guides 5. It should be noted that the number of light guides 5 is not limited to the number described above. Further, more or less than half of the light guides 5 may be connected to the detectors 7. In the present embodiment, the light source 6 is formed by a laser. However, more than one light source may be provided the light of which can be directed in selected light guides 5 by the switch 9.

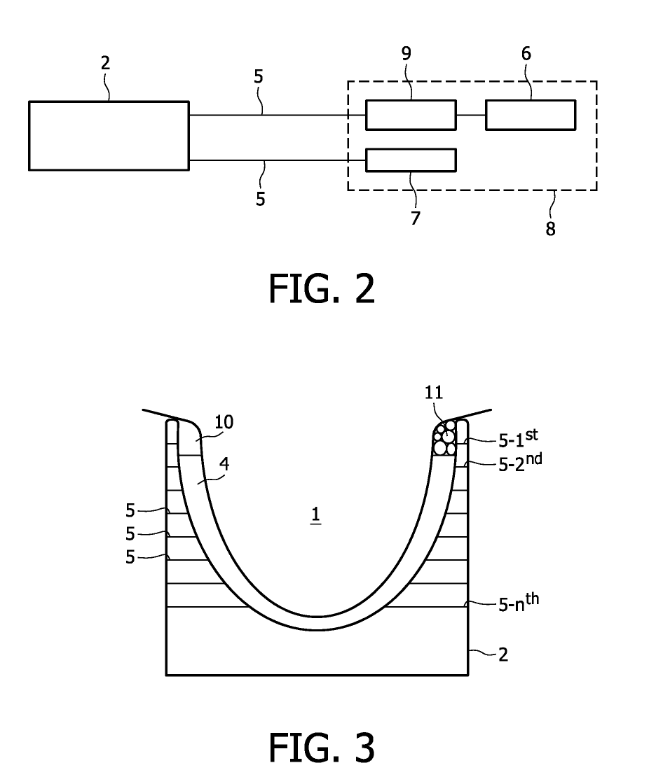

The device is structured such that light from the light source 6 can be subsequently directed to the turbid medium 1 from different directions and light emanating from the turbid medium 1 can be detected by a plurality of detectors 7 the corresponding light guides 5 of which are distributed on the inner surface of the receptacle 2. The device comprises a control unit 8 which reconstructs an image of the interior of the turbid medium 1 based on the signals from the detectors 7. For reconstruction, the signals sampled during a scan in which the light is directed to the turbid medium 1 from different directions are used. For reasons of simplicity, these elements of the device for imaging the interior of a turbid medium are only schematically indicated in FIG. 2. In FIG. 2, the control unit 8 comprises the light source 6 and the plurality of detectors 7. In the embodiment, for example the light from the light source 6 is subsequently directed into different light guides 5 and the light which emanates from the receiving portion 2 in response is detected by the plurality of detectors 7 in each case, e.g. 256 detectors. The size of the receiving portion 2 is such that a space remains between the inner surface of the receiving portion 2 and the turbid medium 1. The receiving portion 2 is structured to receive an optically matching medium 4 for filling a space between an inner surface of the receiving portion 2 and the turbid medium 1. For examination, this space is filled with an optically matching medium 4 which serves to provide optical coupling between the turbid medium 1 to be imaged and the inner surface of the receiving portion 2. The optically matching medium 4 further serves to prevent optical short-cutting between the light guides 5 coming from the light source 6 and the light guides 5 coupling to the detectors 7. Furthermore, the optically matching medium 4 serves to counteract boundary effects in the reconstructed image which are caused by the difference in optical contrast between the interior of the turbid medium 1 and the remaining space in the receiving portion 2. For this purpose, the optically matching medium 4 is provided with optical properties which substantially match the optical properties of the turbid medium 1 to be examined.

In operation, i.e. for imaging the interior of a turbid medium, first a reference measurement with the receiving portion 2 completely filled with the optically matching medium 4 is performed. Thereafter, the turbid medium to be examined, e.g. the female human breast, is immersed in the optically matching medium 4 in the receiving portion 2 and the actual measurement is performed. Both, the reference measurement and the actual measurement consist of a large number of detector signals each forming a measurement set of data. For example, in the embodiment 256×256 detector signals form the measurement set of data, since 256 detectors and corresponding light guides 5 are provided and the light can be directed to the interior of the receiving portion 2 via 256 different light guides 5. In the case that a plurality of different light sources is provided, for example lasers emitting light of different wavelengths, this number of detector signals may be measured for each of the plurality of light sources such that the measurement set of data becomes even larger. The measured detector signals forming the measurement set of data can then be converted into a three-dimensional image of the interior of the turbid medium using a process called image reconstruction. Although the number of measured signals forming the measurement set of data is not restricted to the values described above it becomes clear that a measurement set of data consists of a large number of data and thus requires a considerable time interval.

Since the turbid medium 1 to be examined has to be immersed in the optically matching medium 4 in the receiving portion 2 before the actual measurement is performed, a problem occurs that the space between the receiving portion 2 and the turbid medium 1 may not be completely filled with the optically matching medium 1, as schematically indicated in FIG. 3. For example, a gap 10 filled with air or another gas may be formed in the upper part of the space between the receiving portion 2 and the turbid medium 1, as indicated in the left part of FIG. 3. As another example, gas bubbles 11 may be present in the upper part of the space, as schematically indicated in the right part of FIG. 3. These undesired inhomogeneities 10, 11 in the space between the receiving portion 2 and the turbid medium 1 can lead to artifacts in the reconstructed image or to serious measurement errors if the actual measurement generating the measurement set of data is performed with the inhomogeneities present. In case of the undesired inhomogeneities 10, 11 described above, light will be attenuated less than expected in the region of the inhomogeneities, since, without the inhomogeneities 10, 11 present, the light would pass through the optically matching medium 4. As a result, such inhomogeneities 10, 11 can lead to intensities detected by the respective detectors 7 which are between one and two orders of magnitude higher than the intensities which would be detected without such inhomogeneities present.

In the conventional method, it is particularly disadvantageous that the presence of undesired inhomogeneities will only become clear after the measurement and evaluation of the results, since a large number of data have to be sampled for the measurement set of data.

In case of a (hypothetical) infinitely large diffuse medium, if the logarithm of the intensity measured by the respective detectors 7 multiplied by the distance from the light guide 5 connecting to the light source 6 to the light guide 5 connecting to the detector 7 was plotted in a graph as a function of the distance from the light guide 5 connecting to the light source 6 to the light guide 5 connecting to the detector 7, a linear relation would follow between the quantities in the graph. In the real cup-like geometry of the receiving portion 2, some deviation close to the boundary occurs but, on the whole, the measured points still lie approximately on a straight line. However, if undesired inhomogeneities 10, 11 as described above are present in the light path from the light source 6 to the respective detector 7, this results in clearly higher measured intensity values for the affected detector 7 and thus in a deviation from the expected straight line. Thus, this deviation can be used to identify the presence of an inhomogeneity.

In a mode of operation according to the present embodiment, the fact is used that the undesired inhomogeneities will mainly form in the upper region of the space between the turbid medium 1 and the receiving portion 2. This results from the fact that, in case of the inhomogeneities formed by air or another gas, the inhomogeneities have a lower density as compared to the optically matching medium 4 and thus will float up. As can be seen in the schematic illustration in FIG. 3, the presence of inhomogeneities 10, 11 will mainly affect the detected intensities with respect to light paths involving the rings of light guides 5 on the side of the opening 3 of the receiving portion 2, i.e. involving the upper rings 5-1^{st}, 5-2^{nd}, . . . of light guides 5. According to the embodiment, in order to detect the presence of inhomogeneities, a fast-measurement in which a fast-measurement set of data is sampled which is reduced as compared to the actual measurement set of data is performed before the actual measurement is performed. In other words, a fast-measurement is performed in which a reduced, smaller number of detector signals is sampled in order to determine whether inhomogeneities are present or not.

For example, in the fast-measurement only those light guides 5 connecting to the light source 6 which are located in the upper ring 5-1^{st} of the light guides 5 located close to the opening 3 of the receiving portion 2 are used for irradiating the interior of the receiving portion 2 and the resulting intensities are detected by the plurality of detectors 7. Thus, the fast-measurement generates a fast-measurement set of data which is substantially reduced compared to the actual measurement set of data. In this context “reduced set of data” is understood as a set of data comprising less data values, thus a reduced amount of data.

As a non-restricting example, 16 light guides 5 connecting to the light source 6 are present in the upper ring 5-1^{st} of light guides, 256 light guides 5 connecting to the light source 6 are distributed over the entire receiving portion 2 for the actual measurement, and 256 detectors are provided. In this case, the fast-measurement set of data comprises 16×256 detector signals which is only a small fraction compared to 256×256 detector signals in the measurement set of data. It should be clearly understood that the example given above is only for explanation and that the embodiment is not restricted to these explicitly given values. More generally speaking, instead of X×Y detector values in the measurement set of data, the fast-measurement set of data comprises only x×Y values with x<X or x<<X, respectively.

As an alternative, not only the ring 5-1^{st} of light guides located closest to the opening 3 can be used, but also the second 5-2^{nd} or third ring of light guides seen from the direction of the opening 3. Two or more of these rings of light guides can be used for the fast-measurement. Further, with regard to the respective ring of light guides not necessarily all light guides 5 connecting to the light source 6 have to be used for the fast-measurement.

The amount of data for the fast-measurement set of data can be further reduced if not only for source positions, i.e. light guides 5 connecting to the light source 6, only those of the upper rings 5-1^{st}, are used, but also for the detector positions. In this case, only those detectors connected to light guides 5 ending in the upper rings 5-1^{st} 5-2^{nd} etc. are used during the fast-measurement. Instead of X−Y detector signals for the actual measurement, then the fast-measurement set of data comprises only x×y values with x<X or x<<X and y<Y or y<<Y. Thus, the fast-measurement requires sampling of even less data.

The fast-measurement set of data generated during the fast-measurement is compared to the expected values. If the values of the fast-measurement set of data are clearly higher than the expected values, an undesired inhomogeneity is judged to be present in the space between the turbid medium 1 and the receiving portion 2. As a result, by making the fast-measurement before the actual measurement is started, the presence of undesired inhomogeneities can be detected before the actual measurement is executed which generates a large measurement set of data. If undesired inhomogeneities are detected, they can be easily removed before the actual measurement is started. Thus, precious time and effort can be saved.

Further, the described method can be applied to known devices for examining the interior of turbid media and the known devices can be adapted to perform the described method. Thus, the advantages can be realized in a cost-efficient way.

The realization according to the embodiment can also be achieved by a computer program product which can be loaded in the control unit of a device for imaging the interior of turbid media. In this case, the computer program product is implemented such that, when loaded in the control unit of the device for imaging the interior of turbid media and executed, it causes the device to perform the fast-measurement generating the fast-measurement set of data before the actual measurement is started. Further, the computer program product can be implemented such that the other features described in this specification become realized in the device for imaging the interior of turbid media after loading of the computer program product. The computer program product can be provided on a physical data carrier such as CD-ROM or DVD, for example.

The result of the fast-measurement can be provided to an operator of the device for imaging the interior of turbid media as a graphical representation, e.g. by displaying a plot on an optical user interface such as a monitor. In this case, the operator can easily determine from the graphical representation whether undesired inhomogeneities are present or not. The control unit 8 of the device for imaging the interior of turbid media can be adapted such that it provides the graphical representation. For example, the graphical representation can be a plot showing the logarithm of the intensity measured by the respective detectors 7 multiplied by the distance from the light guide 5 connecting to the light source 6 to the light guide 5 connecting to the detector 7 as a function of the distance from the light guide 5 connecting to the light source 6 to the light guide 5 connecting to the detector 7, as has been described above. If the plot shows a deviation to higher intensities from the expected straight line, it can be judged that an undesired inhomogeneity is present. It should be noted, that an undesired inhomogeneity would result in a hump to higher intensities in such a graphical representation.

If the expected values are also shown in the graphical representation, the undesired inhomogeneity can be easily identified by the operator. Further, the spatial distribution of the measured detected intensities can be graphically represented. This provides spatially resolved information about inhomogeneities. In this case, the operator can identify from the graphical representation at which position or positions an undesired inhomogeneity or undesired inhomogeneities are located. Thus, the inhomogeneities can be removed even faster.

A further mode of operation will now be described. It has been described above that methods for imaging the interior of turbid media are available in which a fluorescent contrast agent is injected in the turbid medium 1. The fluorescent contrast agent is designed such that it accumulates in cancer cells. In the known method, a measurement step generating a measurement set of data for imaging the interior of the turbid medium 1 to be examined is performed after the fluorescent contrast agent has been injected to achieve a static image of the interior of the turbid medium 1. Thus, the spatial distribution of the fluorescent contrast agent in the turbid medium at a certain time-point can be determined. However, due to the fact that only a static image of the spatial distribution of the contrast agent is determined, no information about the dynamic behavior of the contrast agent, i.e. the change of the spatial distribution as a function of time, can be determined. If a plurality of measurement steps generating measurement sets of data were performed, the measurement steps would take a long time. In many cases, this measurement time would exceed the relevant time constants of the dynamic behavior of the contrast agent. Thus, the known method is not suited for determining the additional information which can be gathered from the dynamic behavior of the contrast agent.

It has been described above that a large number of fibers acting as light guides 5 for directing light from the light source 6 into the interior of the receiving portion 2 at different positions are provided in the device for imaging the interior of turbid media, e.g. 256 such fibers. During a measurement step generating a measurement set of data for imaging the interior of the turbid medium 1 to be examined, this large number of light guides 5 is used for subsequently irradiating the turbid medium in order to achieve a large amount of information. However, it has been found that a reasonable scan of the turbid medium 1 allowing the dynamic behavior of the contrast agent to be determined can be performed using a limited number of these light guides 5, for example using only 64 of the light guides 5 for directing light from the light source 6 into the interior of the receiving portion 2. Thus, a fast fast-measurement generating a reduced fast-measurement set of data can be performed with a limited amount of source positions with respect to the turbid medium 1 located in the receiving portion 2. This fast-measurement step can be performed fast enough to determine the dynamic behavior of the contrast agent and thus the additional information contained therein. The detectability of for example lesions in the turbid medium 1 obviously decreases if only the information of the fast-measurement set of data is used, however, the detection limit only changes a few millimeters if the number of source positions is decreased significantly. Thus, the information contained in the fast-measurement set of data is sufficient to determine the dynamic behavior of the contrast agent with sufficient accuracy. Further, a measurement step generating a measurement set of data is performed in order to achieve an image of the turbid medium 1 with high accuracy.

According to an alternative, a plurality of fast-measurement steps generating fast-measurement sets of data is performed. Each of these fast-measurements can be performed fast and the plurality of fast scans allows determining accurately the dynamic behavior of the contrast agent, i.e. the change in its spatial distribution as a function of time.

It has been described above that a switch 9 is provided to subsequently direct the light from the light source 6 into different light guides 5 for irradiating the turbid medium 1. For example, the light guides 5 are formed by light guiding fibers and the switch 9 is a fiber switch. In the switch 9, ends of the light guides 5 connecting to the interior of the receiving portion 2 are arranged. For subsequently switching the light from the light source 6 to different source positions with respect to the turbid medium 1 placed in the receiving portion 2, the light is subsequently directed into different light guides 5 by the switch 9. If only a limited number of the light guides 5 is used during the fast-measurement step, the light has to be switched only to a limited number of positions by the switch 9, for example only 64 light guides out of 256. In this example, if 64 of 256 light guides are used, the light has to be switched to one fourth of the light guides 5 during the fast-measurement step. If every fourth of the light guides 5 arranged in a row was used, the switch 9 would have to be displaced over four light guides 5 in every switching. This would greatly increase the switching time and thus the overall time of the fast-measurement step. In order to overcome this problem, the switch-side ends of the light guides 5 which are used in the fast-measurement step are arranged adjacent to each other side by side in the switch 9. The ends of the light guides 5 not used during the fast-measurement step are not interposed between the ends of the light guides 5 used during the fast-measurement step. Thus, the switching time is reduced significantly and a fast fast-measurement step is made possible.

It should be noted that the embodiment is not limited to the exact numbers mentioned above, in particular more or less than 256 light guides for connecting the light source 6 to the interior of the receiving portion 2 may be provided and that not necessarily one fourth of the light guides 5 has to be used in the fast-measurement step.

Although it has been described that a plurality of light guides 5 is provided in the receiving portion for connecting to the detectors 7, the detectors 7 may also be directly arranged in the receiving portion 2 without light guides 5 interposed. Although a plurality of detectors 7 is described throughout the specification, only one detector 7 may be provided which is connected to a plurality of detection positions in the receiving portion 2, i.e. ends of light guides 5, by means of a switch. However, use of a plurality of detectors 7 is preferred. A less preferred alternative could be providing only one possible source position and a plurality of different detection positions, or providing a plurality of different source positions and only one possible detection position. Further, it should be noted that, in principle, a device comprising only the smaller number of possible source-detector position combinations required for the fast-measurement step can be provided. A device comprising a single light source and a single detector, with at least one of the light source and the detector being movable relative to the receiving portion (2) is also possible. An example of such a device is a device comprising a light source and a detector creating a source position and a detection position which revolve around the receiving portion. At any given time, there is a single light path from the source position to the detection position through the receiving portion, but over the complete measurement and with the source position and the detection position revolving during that measurement, a plurality of different source-detector are positions through the receiving portion are formed over the complete measurement.

The receiving portion 2 does not necessarily have to be formed by a cup-like receptacle as described above. It is also possible to realize the receiving portion 2 by compression surfaces between which the turbid medium is compressed. In this case, the device may comprise an optically transparent, flexible bag for accommodating the turbid medium and the optically matching medium, wherein the flexible bag and its contents, i.e. the turbid medium and the optically matching medium, are compressed between the compression surfaces. In such embodiment the surfaces are arranged mostly in parallel having a plurality of rows of light guides 5. Thus, the construction of the receiving portion could be simplified. Moreover, the position of the surfaces to each other could be made flexible. Thus, by moving the surfaces the receiving portion could be adapted to the size of turbid media.

1. Method of controlling a device for imaging the interior of turbid media; which device comprises:
a receiving portion (2) for receiving a turbid medium (1) to be examined;
at least one light source (6) optically connected to the receiving portion (2) for irradiating the interior of the receiving portion (2); and
at least one detector (7) optically connected to the receiving portion (2) for detecting light emanating from the interior of the receiving portion (2);
the at least one light source (6) and the at least one detector (7) being optically connected to the receiving portion (2) such that a plurality of different source-detector position combinations defining different light paths through the receiving portion (2) are formed over a complete measurement;
wherein the method comprises a fast-measurement step in which a reduced set of data corresponding to only a part of the plurality of source-detector position combinations is generated for providing fast-information about the interior of the receiving portion (2).

2. Method according to claim 1, wherein, in the fast-measurement step, the intensity of light detected by the at least one detector (7) is compared to an expected intensity and, based on this comparison, it is determined whether undesired inhomogeneities (10,11) are present in the receiving portion (2) or not.

3. Method according to claim 2, wherein a graphical representation of the comparison between the detected intensity and the expected intensity is provided to an operator of the device for imaging the interior of turbid media.

4. Method according to claim 3, wherein the graphical representation is provided such that it indicates at which position in the receiving portion an undesired inhomogeneity is present.

5. Method according to claim 1, wherein the fast-measurement step is performed to determine the dynamic behavior of a contrast agent located in the turbid medium (1) or to determine whether undesired inhomogeneities are present in the receiving portion (2).

6. Method according to claim 1, wherein a plurality of fast-measurement steps generating reduced sets of data is performed at different times.

7. Device for imaging the interior of turbid media, the device comprising:
a receiving portion (2) for receiving a turbid medium (1) to be examined;
at least one light source (6) optically connected to the receiving portion (2) for irradiating the interior of the receiving portion (2); and
at least one detector (7) optically connected to the receiving portion (2) for detecting light emanating from the interior of the receiving portion (2);
the at least one light source (6) and the at least one detector (7) being optically connected to the receiving portion (2) such that a plurality of different source-detector position combinations defining different light paths through the receiving portion (2) are formed over a complete measurement;
wherein the device further comprises a control unit (8) which is adapted to control the device such that:
a reduced set of data corresponding to only a part of the plurality of source-detector position combinations is generated in a fast-measurement step for providing fast-information about the interior of the receiving portion (2).

8. Device according to claim 7, wherein the receiving portion (2) comprises a plurality of light guides (5) for optically connecting to the at least one detector (7) and to the at least one light source (6), and wherein the control unit (8) is further adapted such that, during the fast-measurement step, a reduced number of these light guides (5) is used for subsequently directing light into the interior of the receiving portion (2).

9. Device according to claim 7, wherein the control unit (8) is adapted such that, in the fast-measurement step, only light guides (5) located in an upper part of the receiving portion (2) are used for directing light into the receiving portion (2).

10. Device according to claim 8, wherein the plurality of light guides (5) are arranged on the receiving portion (2) in a ring-like structure comprising a plurality of rings (5-1^{st}, 5-2^{nd}, . . . , 5-n^{th}) or sections thereof located in planes perpendicular to a vertical axis (Z), and the control unit (8) is adapted such that, in the fast-measurement step, only upper ones (5-1^{st}, 5-2^{nd}, . . . ) of the rings of light guides are used for directing light into the receiving portion (2).

11. Device according to claim 10, wherein the control unit (8) is adapted such that, during the fast-measurement, only the topmost ring of light guides (5-1^{st}) is used for directing light into the receiving portion (2).

12. Device according to claim 7, wherein the receiving portion (2) is realized in a cup-like form having a plurality of rings (5-1^{st}, 5-2^{nd}, . . . , 5-n^{th}) of light guides (5) or comprises parallel surfaces having rows of light guides (5).

13. Device according to claim 8, wherein a switch element (9) is provided for subsequently directing light from the at least one light source (6) into the light guides (5); and wherein ends of said light guides (5) used during the fast-measurement step, which ends are located in the switch element, are arranged adjacent to each other without ends of light guides (5) not used in the fast-measurement step interposed therebetween.

14. Device according to claim 7, wherein the device is a medical image acquisition device.

15. Computer program product for a device for imaging the interior of turbid media; which device comprises:
a receiving portion (2) for receiving a turbid medium (1) to be examined;
at least one light source (6) optically connected to the receiving portion (2) for irradiating the interior of the receiving portion (2);
at least one detector (7) optically connected to the receiving portion (2) for detecting light emanating from the interior of the receiving portion (2);
the at least one light source (6) and the at least one detector (7) being optically connected to the receiving portion (2) such that a plurality of different source-detector position combinations defining different light paths through the receiving portion (2) are formed over a complete measurement; and
a control unit (8) controlling the operation of the device;
wherein the computer program product is adapted such that, when loaded in the control unit (8), the device is controlled such that:
a fast-measurement step is performed in which a reduced set of data corresponding to only a part of the plurality of source-detector position combinations is generated for providing fast-information about the interior of the receiving portion (2).
